(19) United States
(12) Patent Application Publication
(10) Pub. No.: US 2005/0232894 A1
(43) Pub. Date: Oct. 20, 2005

(54) Antimicrobial skin treatment composition and methods for producing and using an antimicrobial skin treatment composition

(76) Inventors: Gregory Martin Weiner, Omaha, NE (US); Raymond T. Rael, Council Bluffs, IA (US); David G. Wood, Omaha, NE (US)

Correspondence Address: MERCHANT & GOULD PC, P.O. BOX 2903, MINNEAPOLIS, MN, 55402-0903, US

(21) Appl. No.: 10/828,093

(22) Filed: Apr. 20, 2004

(51) Int. Cl. A61K 7/075; A61K 7/08

(52) U.S. Cl. 424/70.28; 514/643

(57) ABSTRACT

The invention relates to an antimicrobial skin treatment composition and methods for producing and using an antimicrobial skin treatment composition. The antimicrobial skin treatment composition includes an antimicrobial package that can provide extended protection against germs and bacteria. The antimicrobial package can include any of the following: a quaternium ammonium antimicrobial agent, a protease enzyme, a preservative, a natural antimicrobial agent, or a lower alcohol. The composition can also include, among other ingredients, a thickening agent, a skin healing ingredient, a humectant, an emollient, a skin moisturizer, or water.

### FIELD OF THE INVENTION

The invention relates to an antimicrobial skin treatment composition and methods for producing and using an antimicrobial skin treatment composition. The antimicrobial skin treatment composition includes an antimicrobial package that provides extended protection against germs and bacteria. The antimicrobial skin treatment composition can be provided with a substantial absence of lower alcohols that have a tendency to deplete skin of its natural oils.

### BACKGROUND OF THE INVENTION

The transmission of bacteria, germs, microorganisms, viruses, and other deleterious substances through skin contact has long been known to be a significant cause of many illnesses. In order to prevent the transmission of such pathogens, there has been a desire to provide an antimicrobial composition that can be applied to a person's hands which kills bacteria and germs and other pathogens.

It is known that some quaternary ammonium agents exhibit antimicrobial action, killing gram positive and gram negative bacteria, as well as viruses and fungi. Numerous antimicrobial compositions including quaternary ammonium agents have been disclosed. See, for example, U.S. Pat. App. No. 2002/0031486 to Lunsmann et al.; U.S. Pat. No. 5,994,383 to Dyer et al.; U.S. Pat. No. 6,087,400 to Dyer et al.; U.S. Pat. No. 5,661,170 to Dyer et al.; U.S. Pat. App. No. 2003/0104018 to Bettle et al.; and U.S. Pat. No. 6,338,855 to Albacarys et al.

Topical antimicrobial compositions often include high levels of lower alcohols, specifically ethanol. See U.S. Pat. No. 6,344,218 to Dodd et al. and U.S. Pat. App. 2002/0176879 A1 to Dodd et al. Compositions high in lower alcohol content can damage skin by causing dryness, cracking, and general discomfort. In addition, composition involving high volumes of lower alcohols have a tendency to remove lipids from the skin that protect against bacterial infection, thereby increasing chances of the transmission of bacteria upon prolonged use of the alcohol-based composition. See Dyer et al., Testing a New Alcohol-Free Hand Sanitizer to Combat Infection, AORN Journal, August 1998, 68(2):239-251. In addition, compositions high in lower alcohol content involve flammability concerns.

Alcohol-free antimicrobial compositions are available. See U.S. Pat. App. No. 2002/0031486 to Lunsmann et al.

### SUMMARY OF THE INVENTION

The present invention relates to an antimicrobial skin treatment composition and methods for producing and using an antimicrobial skin treatment composition. The treatment composition can be applied to the hands to provide effective antimicrobial protection. The composition can be used by anyone desiring to prevent the spread of microbial pathogens, such as health care workers, restaurant employees, and the public in general.

The treatment composition can include an antimicrobial package. This antimicrobial package can include a variety of ingredients, such as quaternary ammonium antimicrobial agent, protease enzyme, preservative, natural skin antimicrobial, and lower alcohol. In addition, the composition can include other beneficial ingredients that moisturize, condition, or otherwise improve the health of skin. Some of these additional ingredients include: thickening agent, skin healing agent, emollient, moisturizer, humectant, and water.

The present invention further includes a process for producing an antimicrobial skin treatment composition and a method of using an antimicrobial skin treatment composition to provide a reduction in the presence of antimicrobial pathogens.

### DETAILED DESCRIPTION OF THE PREFERRED EMBODIMENT

The invention is directed at an antimicrobial skin treatment composition and methods for producing and using an antimicrobial skin treatment composition. It should be understood that the phrase “antimicrobial skin treatment composition” can be referred to more simply as the treatment composition or as the composition. The treatment composition can be applied to a user's hands and rubbed into the skin to provide protection against bacteria, germs, microorganisms, viruses, etc. that may cause illness. The treatment composition can include an antimicrobial package that remains on the skin after the composition has been rubbed into the skin to provide antimicrobial activity. Additional components that can be included in the treatment composition include thickening agent, skin healing agent, emollient, moisturizer, humectant, and water.

The treatment composition can be provided with or without lower alcohols. In general, lower alcohols are considered to be those alcohols that may possess antimicrobial activity but may have a tendency to deplete oils present in the skin and can be characterized as having five or fewer carbon atoms. Exemplary lower alcohols include ethanol, propanol, butanol, and pentanol, and can include straight or branched chains such as n-propanol and isopropanol. When the treatment composition is characterized as being substantially free of lower alcohols, it should be understood that the treatment composition is free of lower alcohol or contains an amount of lower alcohol that is sufficiently small such that repeated use of the treatment composition will generally not cause depletion of oils from skin and result in cracking or dryness of the skin. Characterizing the treatment composition as being substantially free of lower alcohol refers to an amount of lower alcohol, if present at all, at a level of less than about 0.5 wt. % of the composition. Alternatively, a lower alcohol can be included in the treatment composition if it is desired to provide additional antimicrobial properties. When it is desirable to include a lower alcohol in the treatment composition, the treatment composition can comprise from about 20 wt. % up to about 65 wt. % lower alcohol.

A reduction in the level of lower alcohols enables repetitive users, such as workers in the health care industry, to apply the composition on a highly repetitive basis without developing dry and cracked skin. Consequently, repetitive users will be more incentivized to use the treatment composition. Moreover, by maintaining the skin in a healthy condition, it is expected that the skin's natural barrier and defenses against microbial pathogens can continue to thrive. Thus, the composition can contain ingredients which promote the development of healthy skin.

The treatment composition can be characterized as rinse free. That is, the composition can be rubbed into the skin to disperse the composition with no water rinsing required. This enables the composition to exert continued antimicrobial activity. It also allows users to clean and moisturize their skin on-the-go. Furthermore, the treatment composition can be used in places where water is not available. It is pointed out that antimicrobial compositions that are presently available often include surfactants for cleansing. When these compositions are intended to be rinsed off, they can be characterized as cleansing compositions because their function is to clean the skin and not leave a component on the skin to provide for antimicrobial properties.

Antimicrobial Package

The treatment composition includes an antimicrobial package for providing antimicrobial properties. The antimicrobial package can include different antimicrobial components which, acting together, provide a desired level of antimicrobial activity and a desired level of extended protection. The phrase “extended protection” refers to the continued antimicrobial activity of the treatment composition after application of the treatment composition to skin. It is believed that portions of the antimicrobial package will remain on the surface of the skin to assist in providing antimicrobial protection until washed or worn away. It is desirable for the treatment composition to provide an antimicrobial activity characterized by a three log reduction of gram positive bacteria for at least 4 hours after application. Furthermore, it is desirable for the treatment composition to provide antimicrobial activity characterized by a three log reduction of gram positive bacteria for up to about 60 hours after application.

The antimicrobial package includes at least one quaternary ammonium antimicrobial agent, and can include additional components such as a protease enzyme, a preservative, a natural skin antibiotic, and a lower alcohol. It should be understood that the antimicrobial package will include at least one quaternary ammonium antimicrobial agent and the remaining components can be considered optional. That is, the treatment composition can include 0 amount of any one or more of the protease enzyme, the preservative, the natural skin antimicrobial component, and the lower alcohol antimicrobial component.

1. Quaternary Ammonium Antimicrobial Agent

The antimicrobial package can include a quaternary ammonium antimicrobial agent that provides activity against bacteria, germs, microorganisms, and other deleterious substances when provided on skin tissue. The quaternary ammonium antimicrobial agent is preferably provided as one that does not cause excessive irritation to a user's skin. Exemplary quaternary ammonium antimicrobial agents that can be used according to the invention include benzethonium chloride (BZT), benzalkonium chloride, methyl benzethonium chloride, and benzoxonium chloride. A preferred quaternary ammonium antimicrobial agent is benzethonium chloride as this component has been found to provide effective antimicrobial activity while causing only a reduced level of skin irritation, if any at all. In addition, it is believed that the structural characteristics of the BZT molecule render it less susceptible to anionic attack than other quaternary ammonium antimicrobial agents.

It is generally believed that the antimicrobial activity of benzethonium chloride and other quaternary antimicrobial ammonium agents have a tendency to be reduced by the presence of anionic components, such as anionic surfactants. By providing the treatment composition as a cationic system, it is possible to stabilize the benzethonium chloride and thereby preserve the antimicrobial activity of the treatment composition. It should be understood that a “cationic system” can be characterized as substantially free of anionic components.

The treatment composition can include the quaternary ammonium antimicrobial agent in an amount sufficient to provide desired efficacy against bacteria, germs, microorganisms and other deleterious substances that may be present on skin tissue, but should not be provided at a level that causes excessive skin irritation. In general, the amount of quaternary ammonium antimicrobial agent that can be included in the treatment composition can be controlled by government regulation, and is limited by toxicity concerns. Enough antimicrobial agent should be present to exert antimicrobial activity. Accordingly, the treatment composition can include from about 0.1 wt. % and up to about 0.5 wt. % of the quaternary ammonium antimicrobial agent based on the treatment composition. The composition can comprise from about 0.2 wt. % and about 0.4 wt. % quaternary ammonium antimicrobial agent. The treatment composition can comprise about 0.21 wt. % to about 0.3 wt. % quaternary ammonium antimicrobial agent. A treatment composition including benzethonium chloride as the quaternary ammonium antimicrobial agent, can comprise between about 0.1 wt. % to about 0.5 wt. % benzethonium chloride. The treatment composition can also comprise about 0.21 wt. % to about 0.3 wt. % benzethonium chloride.

2. Protease Enzyme

The treatment composition can include a protease enzyme to protect against spores that may be present on a person's hands. Although spores can be removed by regular hand washing, it is expected that some people may not wash their hands sufficiently to remove all the spores while others may not even wash their hands. In addition, certain environments, such as that encountered by caretakers who handle diapers or assist incontinent, elderly people, involve increased exposure to spores. It is believed that protease enzymes may function by attacking the shell of a spore, thereby allowing the quaternary ammonium antimicrobial agent to attack microbes contained within the spore.

Protease enzymes that can be used in the treatment composition include any protease enzyme that will attack a spore's shell, will become active on human skin tissue, and will be compatible with human skin tissue. It is expected that suitable protease enzymes may become active when a person rubs his or her hands together. Accordingly, the enzyme can be activated at a temperature of about 104° F. By way of example, it has been found that the protease enzyme sold under the trade name Alcalase by Novozymes is suitable for use in the composition.

To provide a more effective composition, the enzyme may be coated using a polymer that is compatible with water-based systems, such as carbomers or acrylics. It should be appreciated that the component used to coat the enzyme may change when the composition contains certain additional components, such as a lower alcohol. It is believed that the polymer can serve to maintain the protease enzyme in a neutral state until activation is desired. It is also believed that the polymeric coating can be removed when the skin is rubbed together to render the enzyme more susceptible to activation.

When included, a level of protease enzyme sufficient to attack spores should be incorporated in the composition. The composition can comprise more than about 0.02 wt. % and up to about 1.5 wt. % protease enzyme. The composition can comprise about 0.2 wt. % to about 0.5 wt. % protease enzyme, if protease enzyme is used.

3. Preservative

In order to provide a treatment composition with a longer shelf-life, the composition can include a preservative. While certain quaternary ammonium active ingredients, such as BZT, can exert preservative activity, the composition can include additional preservatives to allow the active ingredient to provide longer-lasting antimicrobial activity. The Applicants believe that certain preservatives exert a synergistic effect with the QAC antimicrobial agent.

It is believed that certain preservative components provide an additive, and even possibly a synergistic antimicrobial effect. Some preservative components which it is believed produce such a synergistic effect include: methylchloroisothiazolinone, methylisothiazolinone, methyl paraben, propyl paraben, imidazoyldinyl urea, diazolidinyl urea, iodopropynyl butylcarbamate, and other preservative formulations such as those manufactured by Sutton Laboratories under the names Germall 115 (imidazolidinyl urea), Germall II (diazolidinyl urea), Germall Plus (a blend of 98% diazolidinyl urea and 2% Iodopropynyl butylcarbamate), Germaben II (a blend of 30% diazolidinyl urea, 11% methyl paraben, 3% propylparaben, and 56% propylene glycol), and Germaben II-E (a blend of 20% diazolidinyl urea, 10% methyl paraben, 10% propylparaben, and 60% propylene glycol). The preservatives methylchloroisothiazolinone and methylisothiazolinone, which can be obtained from Rohm & Haas under the brand name Kathon™ CG, have been found to be effective. The composition can include preservatives having a quaternary structure, as preservatives that do not have such a structure could potentially reduce the efficacy of the antimicrobial agent.

When a preservative is employed, the composition should include at least the amount of preservative necessary to obtain a preservative effect. However, some preservatives can be potentially harmful at high concentrations. This is a greater concern in leave-on compositions (compositions that are not rinsed off). Accordingly, the treatment composition can comprise from about 0.01 wt. % and up to about 0.2 wt. % preservative. The composition can also comprise from about 0.05 wt. % to about 0.1 wt. % preservative.

4. Natural Skin Antimicrobial Component

The treatment composition can include a natural antimicrobial component to help provide antimicrobial activity. A natural antimicrobial component can be characterized as a component found in skin tissue that provides natural antimicrobial activity. An exemplary natural antimicrobial component is squalane. Squalane is a fatty substance and has been shown to replenish damaged skin by both protecting against microbial pathogens and by moisturizing the skin. Natural skin antimicrobial components include, among others, licorice root extract (glycyzrrhiza Glabra) and azealic acid.

When used, a quantity of the naturally occurring antimicrobial component sufficient to obtain antimicrobial activity should be included. The upper limit on the amount of the natural skin antimicrobial component can be selected by recognizing that levels beyond a certain amount fail to provide additional advantages and result in unnecessary expenditure. The treatment composition can include at least about 0.01 wt. % natural skin antimicrobial component, and can include less than about 3.0 wt. % natural skin antimicrobial component. The composition can also comprise from about 0.1 wt. % to about 0.5 wt. % natural skin antimicrobial agent. When squalane is used as the natural skin antimicrobial component, it can be provided in the composition in an amount of at least about 0.1 wt. % and less than about 1 wt. %. The composition can comprise from about 0.3 wt. % to about 0.7 wt. % squalane. Other natural skin antimicrobial agents may require different concentrations. For example, the composition can comprise from about 0.01 wt. % to about 0.02 wt. % licorice root extract.

5. Lower Alcohol Antimicrobial Component

As previously mentioned, the antimicrobial package can optionally include a lower alcohol. Lower alcohols exhibit strong antimicrobial activity, and can be used to provide a treatment composition that affords a broader scope of antimicrobial protection. It is believed that alcohols provide stronger antimicrobial action against viruses and certain gram negative bacteria than some other antimicrobial components exhibit.

Many current alcohol based compositions include 59-62 wt. % alcohol in order to provide sufficient antimicrobial activity. Treatment compositions of the present invention including similar levels of alcohol can be prepared. By virtue of the antimicrobial package, however, lower levels of lower alcohols can be included without forfeiting the antimicrobial activity of the alcohol. For instance, the composition can include about 20-30 wt. % lower alcohols and yet realize a level of antimicrobial activity obtainable in a composition comprising 60 wt. % or more alcohol when the antimicrobial package is absent. A reduction in the volume of lower alcohol helps to maintain healthier skin while also reducing flammability risks posed by compositions including higher concentrations of lower alcohols.

It is believed that reducing the volume of alcohol to obtain the antimicrobial action of alcohol can be attributable to a combined effort between an antimicrobial agent, such as a quaternium ammonium antimicrobial agent, and the alcohol. Without intending to be bound by theory, it is believed that the cationic charge of a quaternium ammonium antimicrobial agent attacks the bacterial membrane, while the alcohol denatures the proteins contained inside the bacteria.

As previously mentioned, lower alcohols that can be used include straight or branched alcohols having about 5 carbon atoms or less. Some examples include, methanol, ethanol, and propanol. Ethanol is a preferred lower alcohol since it reduces the risks of harm resulting from accidental ingestion and other toxicity concerns.

Extremely high levels of alcohol involve toxicity concerns. Moreover, it is believed that alcohol begins to lose antimicrobial activity at extremely high concentrations because it requires water to exert antimicrobial activity. Accordingly, the composition can comprise from about 20 wt. % up to about 65 wt. % lower alcohols. The composition can comprise from about 25 wt % to about 62 wt. % lower alcohol, or even about 20 to about 30 wt. % when lower alcohol is included.

Of course the composition can also be provided substantially free of alcohol. For instance, the composition can comprise less than about 0.5 wt. % lower alcohol. The composition can also include less than about 0.25 wt. % lower alcohol. In fact, the composition can be completely prepared completely free of lower alcohols.

Thickening Agent

The viscosity of the treatment composition can be adjusted by including a thickening agent to provide the desired viscosity. In general, consumers disfavor compositions that are runny. In addition, increasing the viscosity of the composition allows a user to more evenly spread or disperse the composition over the skin tissue.

Thickening agents that can be used in the treatment composition are preferably those that do not adversely affect one or more of the antimicrobial agents. Because the quaternary ammonium antimicrobial components exhibit a positive charge, it is expected that anionic thickening agents may have a tendency to adversely affect the quaternary antimicrobial agent. Thus, nonionic or cationic thickening agents can be used to avoid any potential inhibition of the quaternary antimicrobial agent.

Exemplary thickening agents that can be included in the treatment composition include higher fatty alcohols. It has been found that these agents can be used without encountering a phase stability problem. Moreover, it has been discovered that fatty alcohols can be used as thickening agents without detracting from the efficacy of quaternary ammonium antimicrobial agents. The term “fatty alcohol” as used herein, refers to components of the formula R—OH wherein R is a branched or straight chain alkyl group having at least 11 carbon atoms. For example, this category of components includes stearyl alcohol, cetyl alcohol, cetearyl alcohol, lauryl alcohol, behenyl alcohol, oleyl alcohol, and the like, but is not intended to be limited thereto. It should be noted that the term fatty alcohols refers to alcohols that are not lower alkyl alcohols, such as ethanol, which have been used as antimicrobial agents in prior art compositions and which can cause dryness and irritate the skin.

Other thickening agents that can be used include reacted fatty alcohols such as cetyl myristate; polyethylene glycol esters, such as polyethylene glycol monostearate and polyethylene glycol distearate such as those available under the names polyethylene glycol 150 monostearate and polyethylene glycol 150 distearate. In addition, the thickening agent can comprise carbomers. Carbomers are water immiscible components that can be used to form an emulsion. It is believed that carbomers can include polyacrylic acid. Exemplary carbomers are available under the names Carbopol 940 and Carbopol ETD 2020 sold by Noveon, and Acritamer 940 and Acritamer 941 sold by Rita. The thickening agent can also be a wax, such as a cationic wax. Exemplary cationic waxes that can be used in the treatment composition include those available under the names Quaternium-21 through Quaternium-91. The thickening agent can also include cetrimonium methosulfate (such as the composition sold under the name Crodazosoft DBQ by Croda Inc., located in Penn.).

The thickening agent can comprise a blend of components. For instance, the Applicants have found that a blend of cetearyl alcohol, Quaternium-91, and cetrimonium methosulfate (such as the composition sold under the name Crodazosoft DBQ by Croda Inc., located in Penn.) offers both thickening and skin conditioning qualities. It is believed that this blend has been used in hair conditioning compositions. The Quaternium-91 used in this composition has two dibehenyl chains (fatty moieties) attached to an imidazoline moiety. This cationic wax blend is believed to soften and condition the skin. By using a wax that is cationic in nature, it is believed the wax can be used without risking any deactiviation of the quaternary ammonium antimicrobial agent. It is believed that other hair conditioning agents, such as those containing waxes, or cationic waxes, could be used in the composition as well. For example, the composition can also contain a cationic hair conditioning composition as a thickening agent.

Thickening agents can provide a variety of characteristics desirable in an antimicrobial composition. When prepared under the method discussed infra, these components can provide a sufficiently viscous composition that remains phase stable for an extended period of time, usually one to three years, preferably three years. Another function served by the thickening agents is moisturization. For instance, fatty alcohols act as skin conditioning agents or emollients, refreshing skin that may be irritated by repetitive washing, especially when using a composition including antimicrobial agents and/or alcohols. The thickening agent can also assist in preventing the loss of moisture from the skin.

It is contemplated that the treatment composition disclosed herein can be used in multiple types of formulations. Enough thickening agent should be present to provide a composition having a thickness that is desirable to consumers, which will vary depending upon the form of the composition. Thus, the composition can have a viscosity of about 10 centepoise (cps) (using a Brookfield viscometer, spindle 4 at 10 rpm) or more. Regardless of the format employed, the composition should not be excessively thick, as it will not properly disperse the active ingredients when applied to the hands and will not adequately dispense from a dispenser. For this reason, the composition can have a viscosity of less than about 18,000 cps.

It should be understood that it may be possible to exclude the thickening agent from the composition if the composition has a viscosity that is desirable for a given application without the presence of any thickening agent.

The composition can be provided as a spray. When the composition is provided in this form, the viscosity can be from about 10 cps to about 1,000 cps to assist in delivery as a spray. The composition can be provided having a viscosity from about 20 cps to about 500 cps, and can be provided having a viscosity from about 30 cps to about 200 cps.

The treatment composition can also be provided as a crème or lotion. When provided in this form, the viscosity of the composition can be about 9,000 cps to about 18,000 cps, and about 11,000 cps to about 14,000 cps. The treatment composition can also have a viscosity from about 12,500 cps to about 13,500 cps. It should be understood that the spray and lotion are two exemplary formats in which the compositions can be provided. It should be understood that the treatment composition can be provided in alternative forms or formats.

The amount of thickening agent can vary depending on the desired viscosity. For instance, when the composition is provided as a crème or lotion the composition can comprise from about 3 wt. % to about 7 wt. % thickening agent. The crème composition can comprise from about 4.5 wt. % to about 6.0 wt. % thickening agent. Persons of ordinary skill in the art will recognize that these quantities will vary depending upon the additional ingredients used in the composition.

Alternatively, when the composition is provided as a spray, the amount of thickening agent can be reduced. In fact, the thickening agent may be completely absent, if desired, when the composition is provided as a spray. If thickening agent is included, a spray-form composition can comprise from about 0.01 wt. % to about 1.0 wt. % thickening agent. The composition can also comprise about 0.05 wt. % to about 0.5 wt. % thickening agent. Again, the concentration of thickening agent will vary depending upon the desired format of the composition.

Skin Healing Agent

To aid in developing healthy, well moisturized skin, the composition can further contain a skin healing agent. Numerous skin healing agents can be used. By way of example, aloe vera can be included in the treatment composition. Aloe vera offers many benefits for the care of skin. It has been used to treat sunburn and otherwise heal damaged skin for many years. It provides a soothing sensation when applied to the skin and increases the likelihood that highly repetitive users, such as health care workers, will use the composition as frequently as recommended. Thus, aloe vera can help maintain and replenish nutrients vital to the skin barrier. In addition, aloe vera contains anti-oxidants. It is believed that anti-oxidants react with free radicals to prevent the formation of age related diseases such as cancer. Some of the skin healing agents other than aloe vera which may be used include various forms vitamin E, such as D-alpha tocopheryl acetate, DL-alpha tocopherol acetate, and mixed tocopheryls; alantoin; thiocitic acid; and apricot kernal (prunis Armeniaca).

The skin healing agent is an optional component of the treatment composition. When the skin healing agent is included in the skin treatment composition, it should be included in an amount sufficient to obtain the desired benefits of the skin healing agent. It is expected that beyond a certain level or concentration, increased amounts of the skin healing agent will have little or no additional effect. It is believed that the treatment composition can include at least about 0.01 wt. % skin healing agent, and can include less than about 15 wt. % skin healing agent. The treatment composition can include about 2 wt. % to about 10 wt. % skin healing agent.

Certain skin healing agents require less amounts than others to be effective components. For instance, vitamin E is a rather potent skin healing agent. Since this is an expensive component, excessive concentrations can cause unnecessary expenditures. Accordingly, the treatment composition can comprise from about 0.01 wt. % to about 2.0 wt. % vitamin E. Other components, such as reconstituted aloe vera, are not as potent. Thus, the composition can comprise from about 1 wt. % to about 15 wt. % aloe vera. The composition can also comprise from about 2 wt. % to about 10 wt. % aloe vera. In the case of aloe vera, it should be understood that reconstituted aloe vera can generally be targeted as having a concentration or solids level that is relatively equivalent to aloe vera that has not been reconstituted. In addition, reconstituted aloe vera can be obtained from a powder such as aloe vera gel powder 200:1 from AgroMayl Bota nica S.A.DEC.V. It is understood that reconstituting the aloe vera gel powder at a ratio of water to powder of 200:1 will result in a reconstituted aloe vera product having about 0.5 wt. % total solids. While the aloe vera can be available as a powder to provide reconstituted aloe vera, it should be understood that other sources of aloe vera can be utilized according to the invention.

Emollients and Moisturizers

In order to maintain healthy skin, and replenish any skin that is irritated by the antimicrobial agent, the composition can include emollients and moisturizers. These emollients and moisturizers can be trapped in micelles created by a thickening agent, or nonionic or cationic surfactant, and released upon contact with the skin. For example, the composition can include silicon, glycerin, petroleum, paraffin, beeswax, lanolin, cod liver oil, elastin, olive oil, coconut oil, avocado oil, castor oil, peanut oil, sesame oil, and almond oil, light mineral oil, vegetable oil, cocoa butter, or alantoin. Silicon also forms a barrier to prevent irritants, bacteria, grime, or other chemicals present in the environment from penetrating the skin and overwhelming the antimicrobial package. In order to achieve these advantages, the composition can further include light mineral oil and/or silicone containing components such as polysiloxane.

The emollient and moisturizer component can be optional in the treatment composition, and can be included in the treatment composition at varying amounts depending upon the desired level of moisturization. When the treatment composition includes an emollient and moisturizer, it can be included in an amount of at least about 0.2 wt. %, and can be included in amount of less than about 5 wt. %. The treatment composition can comprise about 1 wt. % to about 3 wt. %, or about 1.5 wt. % to about 2.5 wt. %, emollient and/or moisturizer.

Humectants

Humectants enable the skin to hold more moisture, thereby promoting the action of the moisturizers and emollients. Accordingly, the antimicrobial composition can contain humectants. This is a common ingredient in hand washes, and those having ordinary skill in the art would recognize that many different humectants could be used. For example, the following humectants could be used in the composition: glycols, propylene glycol, glycerin, sorbitol, urea, alpha hydroxy acids (AHAs), and sugars. A preferable humectant is propylene glycol. The humectant component is optional. When the treatment composition includes a humectant, it can be included in an amount of at least about 0.5 wt. % and it can be provided in an amount of less than about 5 wt. %. The treatment composition can comprise about 2 wt. % to about 4 wt. % humectant.

Water

The treatment composition can include water as a carrier and/or diluent. In addition, the treatment composition can be characterized as water-based. Many of the components of the treatment composition can be mixed with water prior to formulation of the treatment composition. Accordingly, the characterization of the amount of water in the treatment composition refers to total water content resulting from added water and water present in other components. The amount of water will vary depending upon the format of the composition and whether any lower alcohols are included in the composition. For instance, when the composition is intended to be used as a spray, the water content can be higher. Conversely, when a lotion or cream is desired, or when alcohol is used, the water content can be replaced with other constituents. For example, when lower alcohols are included in the composition, the composition can include at least about 15 wt. % water and can include less than about 80 wt. % water. When the treatment composition is substantially free of lower alcohols, the composition can include at least about 65 wt. % water and can include less than about 97 wt. % water.

In addition, the composition can be characterized as free of anionic components that inhibit the antimicrobial effect of the antimicrobial agents preferably employed in the composition. As previously mentioned, it is theorized that anionic components may inhibit the activity of the cationic quaternary ammonium antimicrobial agent. Accordingly, if desired, the composition can be produced substantially free of anionic components. For instance, the composition can comprise less than about 2 wt. % anionic components, or even less than about 0.5 wt. % anionic components. Of course, the composition can be completely free of anionic components as well.

Moreover, the treatment composition can be provided substantially, or even completely, free of nonionic, anionic, or amphoteric surfactants. It should be understood that surfactants include compounds generally used by those of skill in the art specifically for their ability to reduce surface tension in antimicrobial compositions and provide foaming or cleansing action. It should be understood that by “surfactant” the Applicants do not intend to refer to compounds that are not typically used for their surfactant properties, such as fatty alcohols and quaternary ammonium antimicrobial compounds.

For instance, nonionic surfactants that can be excluded from the composition include reaction products such as those formed by the reaction of compounds such as aliphatic alcohols, acids, amides, or alkly phenols with compounds such as alkylene oxides, including ethylene oxide and propylene oxide. Other nonionic surfactants that can be excluded include: the condensation products of alkyl phenols and alkylene oxides; the condensation products of aliphatic primary or secondary, linear or branched, alcohols with alkylene oxides; as well as the products made by condensation of ethylene oxide with the reaction products of propylene oxide and ethylenediamine. Additional nonionic surfactants that can be excluded include long chain tertiary amine oxides, long chain tertiary phosphine oxides, dialkyl sulfoxides, and the like. Still other nonionic surfactants that can be excluded from the composition include sugar amides, lactobionamides, as well as alkyl polysaccharides, and glucosides including decyl glucoside, lauryl glucoside, and coco-glucoside. Other nonionic surfactants include octoxylenol and nonoxynol-9. Nonionic surfactants also include cocamidopropyl dimethyl betaines, such as alkanolamine, alkyldimethyl oxide, coconut monoethanolamine, cetyldimethylamine oxide, stearamine oxide, oleamine oxide, and cocamidopropylamine dimethyl oxide; as well as the general class of alkamine oxide surfactants. Additional nonionic surfactants include those discussed in U.S. Pat. No. 5,389,279, U.S. Pat. No. 5,009,814, U.S. Pat. No. 3,723,325, U.S. Pat. No. 4,565,647, and U.S. Pat. App. No. 2002/0031486. These surfactants can be totally excluded from the composition or can be substantially excluded. It should be understood that substantially excluded refers to the presence of less than about 0.1 wt. % nonionic surfactant, and preferrably less than about 0.01 wt. % nonionic surfactant.

Amphoteric surfactants can be excluded from the composition. Exemplary amphoteric surfactants that can be excluded include cocamidopropyldimethyl betaine, cocamidobetaine, oleyl betaine, cocamphddiacetate, cocamidopropylhyrdoxysultaine, and cocamidopropyldimethyl betaine. Other amphoteric surfactants comprise amphocarboxylates, amidoalkyl sultaines, amphophosphates, phosphobetaines, pyrophosphobetaines, and carboxyalkyl alkyl polyamines. The amphoteric surfactants can be substantially excluded which means that the composition contains less than about 1 wt. % amphoteric surfactant.

The composition can also be produced substantially, or even completely, free of cationic surfactants. Cationic surfactants include fatty acid surfactants. For example, fatty acids having from about 10 to about 32 carbon atoms, such as stearic acid, can be excluded from the composition. Other cationic surfactants that can be substantially excluded from the composition include: cationic surfactants having one or more fatty moieties such as fatty moieties having from about 10 to about 25 carbon atoms. Additional cationic surfactants that can be substantially excluded include ammonium compounds substituted with one or more lower alkyl moiety, and ammonium compounds substituted with at least one fatty moiety. Methyl ammonium chlorides are also cationic surfactants, such as cetyl trimethyl ammonium chloride, trimethyl coco quaternary ammonium chloride, diquaternary polydimethylsiloxane, trimethyl quaternary ammonium chloride. The composition can comprise less than about 5 wt. % cationic surfactant. The composition can comprise less than about 1 wt. % cationic surfactant. In fact, the composition can be free from nonionic surfactant such as the cationic surfactant discussed above.

### Method for Preparation of the Composition

It is possible for the preparation of the composition to begin by diluting some of the components of the composition with water. For instance, skin healing agents can be diluted or reconstituted in water. By way of example, dry form aloe vera can be used, wherein the concentrated, dry form aloe vera can be reconstituted in water. Of course, other skin healing agents in various forms can also be used. In addition, any other ingredients which will not degrade, boil off, or otherwise be harmed by elevated temperatures can also be added in this step.

This composition can be heated to 158-210° F. (70-99° C.). The mixture can also be heated to 165-185° F. (73-85° C.), or 176° F. (80° C.). It will be understood by those of skill in the art that this temperature can vary depending upon various conditions, such as pressure, and the addition of other components which can affect the colligative properties of the mixture. This temperature range allows for the formation of a phase stable composition as discussed below.

A thickening agent, such as a fatty alcohol, can be added to the heated mixture. Alternatively, the thickening agent can be added prior to heating of the mixture. Either way, the mixture can be brought back to the aforementioned temperature range, and maintained at that temperature, preferably under slight agitation (600-900 rpm), for a period of time sufficient to create a composition having a desired viscosity. Typically, high-speed agitation is avoided as it can cause foaming of the mixture. It has been discovered that by allowing the mixture to be maintained within this temperature range for a sufficient period of time, a composition including a thickening agent can exhibit desirable phase stability and viscosity. When the thickening agent containing mixture is maintained at an elevated temperature until it passes a pearlized stage, the thickening agent can remain phase stable with the water, avoiding the formation of a two-phase composition.

As a result of the heating, the thickening agent can melt into micelles and form an emulsion. When the micelles have been reduced to a sufficiently small size the composition can exhibit the desired phase stability over an extended period of time. Thus, the mixture can be maintained under the above conditions until the mixture contains micelles having a relatively uniform size, to retain the stability of the emulsion. In order to ensure the mixture has been heated for a sufficient period of time, it can be analyzed under a microscope to ensure that the micelles are of a uniform small size. Of course microscopic analysis is not necessary to produce a composition exhibiting adequate phase stability. Once the desired micelle size or viscosity has been observed, the mixture can be chilled. The mixture can be rapidly chilled to ensure that the micelles maintain their uniform, small size. It should be understood that the term “mixture” refers to the composition at any point in the process, including before and after an emulsion has been formed. The term “mixture” can even refer to the final treatement composition. The term emulsion, however, refers to a composition in which an emulsion has already been formed.

When a protease enzyme is included in the composition, the mixture should be chilled to a temperature below the activation temperature of the protease enzyme. It will be appreciated that this temperature may be adjusted in relation to the protease enzyme that is added to the composition. Different enzymes can require different chill temperature points. For example, when the enzyme Alcalase is used, the mixture should be chilled to about 104 degrees Fahrenheit or less.

Although it could be added in a prior or subsequent step of the process, typically the antimicrobial agent is added after the temperature of the mixture has been reduced. In order to facilitate better dispersion of the antimicrobial agent, the antimicrobial agent can be pre-mixed in water prior to addition to the mixture.

The pH of the mixture can be adjusted using a base such as 10% sodium hydroxide. The pH of the mixture can vary, for instance, the pH can be between about 6-7. The pH of the mixture can be about 6.4. While it is not important where in the process the pH is adjusted, it should be at an appropriate level before a protease enzyme is added, if at all, to the mixture.

As stated before, when a protease enzyme is included in the composition, it can be added after the mixture's pH has been appropriately adjusted and the mixture has been reduced to the appropriate temperature. In addition, the protease enzyme can be coated with a polymer prior to adding the enzyme to the mixture to prevent activation until the composition is frictionally heated upon application to the user's skin.

Additional ingredients, such as preservatives, naturally occurring antimicrobial components, skin healing agents, natural skin antimicrobial agent, humectants, emollients, and the like, can be added to the mixture at any time, provided such components do not require certain temperatures or pH levels. For instance, lower alcohols typically have low boiling points. Therefore, when lower alcohols are included in the composition they are typically added only after the composition has been chilled. It is also possible that the mixture is chilled in steps, wherein the lower alcohol is added following a second reduction in temperature.

The following is an example preparation of the composition.

### EXAMPLE 1

Pharmaceutical grade aloe vera powder at a concentration of 200 parts aloe vera to 1 part water (obtained from AgroMayal Botanica S.A. de C.V., but readily available from many suppliers of cosmetic ingredients), was reconstituted in water to provide a composition having the same water to aloe vera ratio naturally present in an aloe vera plant. This composition was mixed and heated to 176° F. (80° C.).

Next stearyl alcohol (here produced by Rita Corp., but commonly available) and Crodazosoft DBQ (produced by Croda Inc., located in Penn., and containing a mixture of cetearyl alcohol, quaternium-91, and cetrimonium methosulfate), were added to the composition in an amount equal to 5.5 wt. % of the end composition. The mixture was brought back to the aforementioned temperature, and maintained at that temperature for 10-15 minutes with slight agitation (600-900 rpm). When the mixture reached a viscosity of 13,000 cps (using a Brookfield viscometer, spindle 4 at 10 rpm), it was analyzed under a microscope to ensure that the micelles were of a uniform small size. Once the desired micelle size had been observed, the mixture was chilled to below 104° F.

Next, propylene glycol USP (Dow Chemical) was added to the emulsion (3.0 wt. %). Squalane (Fitoderm, manufactured by Cognis) was added (0.5 wt. %), Light Mineral Oil 70, USP (Dow Corning 200(R) Fluid, 100CST) was added (2.0 wt. %), and DC 200 Silicon was added (0.25 wt. %). The mixture was agitated during the addition of these ingredients.

A pre-mix of benzethonium chloride (Lonzagard Benz. Chloride USP, FPK-22.046, manufactured by Lonza, Inc.) and water was prepared. BZT was diluted in enough water to provide good dispersion of the pre-mix upon addition to the mixture. The pre-mix was blended until clear, and then added to the mixture. Enough BZT was added to comprise 0.3% by mass of the end composition.

Next, the preservative, Kathon™ CG (available from Rohm & Haas) was added to the mixture. Enough Kathon™ CO was added to comprise 0.05 wt. % of the end composition. The pH of the mixture was then adjusted to 6.4, using 10% sodium hydroxide.

Finally, the protease enzyme (Alcalase 2.4L, available from Novozymes North America, Inc.) was added in amount equal to 0.20 wt. % of the composition.

The invention has been described with reference to various specific and preferred embodiments and techniques. However, it should be understood that many variations and modifications may be made while remaining within the spirit and scope of the invention as defined by the claims.

The above specification, examples and data provide a complete description of the manufacture and use of the composition of the invention. Since many embodiments of the invention can be made without departing from the spirit and scope of the invention, the invention resides in the claims hereinafter appended.

1. An antimicrobial skin treatment composition comprising:
(a) an antimicrobial effective amount of an antimicrobial package comprising:
(i) quaternary ammonium antimicrobial agent comprising at least one of benzethonium chloride, benzalkonium chloride, methyl benzethonium chloride, or benzoxonium chloride;
(b) thickening agent comprising a fatty alcohol having at least 11 carbon atoms; and
(c) water, wherein the thickening agent are mixed to provide an emulsion.

2. An antimicrobial skin treatment composition according to claim 1 wherein the composition comprises less than about 0.5 wt. % of lower alcohol having 5 carbon atoms or less.

3. An antimicrobial skin treatment composition according to claim 1, wherein the quaternary ammonium antimicrobial agent comprises benzethonium chloride and the composition comprises about 0.1 wt. % to about 0.5 wt. % of benzethonium chloride.

4. An antimicrobial skin treatment composition according to claim 1, wherein the fatty alcohol comprises at least one of stearyl alcohol, cetearyl alcohol, or cetyl alcohol.

5. An antimicrobial skin treatment composition according to claim 4, wherein the composition comprises between about 3 wt. % and about 7 wt. % fatty alcohol.

6. An antimicrobial skin treatment composition according to claim 1, wherein the antimicrobial package comprises a natural antimicrobial agent comprising at least one of squalane, licorice root extract, or azealic acid, and the composition comprises about 0.01 wt. % to about 3 wt. % natural antimicrobial agent.

7. An antimicrobial skin treatment composition according to claim 6, wherein the natural antimicrobial agent comprises squalane and the composition comprises about 0.1 wt. % to about 1 wt. % squalane.

8. An antimicrobial skin treatment composition according to claim 1, wherein the composition is provided as a crème having a viscosity of about 9,000 cps to about 18,000 cps.

9. An antimicrobial skin treatment composition according to claim 1, wherein the composition is provided as a spray having a viscosity of about 10 cps to about 1,000 cps.

10. An antimicrobial skin treatment composition according to claim 1, further comprising about 0.01 wt. % to about 15 wt. % skin healing agent, wherein the skin healing agent comprises at least one of D-alpha tocopheryl acetate, DL-alpha tocopherol acetate, mixed tocopheryls; alantoin; thiocitic acid; or apricot kernal.

11. An antimicrobial skin treatment composition according to claim 1, further comprising about 0.2 wt. % to about 5 wt. % emollient, wherein the emollient comprises at least one of silicon, glycerin, petroleum, paraffin, beeswax, lanolin, cod liver oil, elastin, olive oil, coconut oil, avocado oil, castor oil, peanut oil, sesame oil, and almond oil, light mineral oil, vegetable oil, cocoa butter, or alantoin.

12. An antimicrobial skin treatment composition according to claim 1, further comprising about 0.5 wt. % to about 5 wt. % humectant, wherein the humectant comprises at least one of glycol, propylene glycol, glycerin, sorbitol, urea, alpha hydroxy acid, or sugar.

13. An antimicrobial skin treatment composition according to claim 1 wherein the composition comprises about 20 wt. % to about 65 wt. % of lower alcohol having 5 carbon atoms or less.

14. An antimicrobial skin treatment composition comprising:
(a) an antimicrobial effective amount of an antimicrobial package comprising:
(i) quaternary ammonium antimicrobial agent; and
(ii) protease enzyme;
(b) thickening agent; and
(c) water, wherein the thickening agent and the water are mixed to provide an emulsion.

15. An antimicrobial skin treatment composition according to claim 14 wherein the composition comprises less than about 0.5 wt. % of lower alcohol having 5 carbon atoms or less.

16. An antimicrobial skin treatment composition according to claim 14, wherein the quaternary ammonium antimicrobial agent comprises benzethonium chloride and the composition comprises about 0.1 wt. % to about 0.5 wt. % benzethonium chloride.

17. An antimicrobial skin treatment composition according to claim 14, wherein the composition comprises between about 3 wt. % and about 7 wt. % thickening agent.

18. An antimicrobial skin treatment composition according to claim 14, wherein the thickening agent comprises a fatty alcohol having greater than 11 carbon atoms.

19. An antimicrobial skin treatment composition according to claim 18, wherein the fatty alcohol comprises at least one of stearyl alcohol, cetearyl alcohol, cetyl alcohol, or mixtures thereof.

20. An antimicrobial skin treatment composition according to claim 14, wherein the antimicrobial package comprises a natural antimicrobial agent comprising at least one of squalane, licorice root extract, or azealic acid, and the composition comprises about 0.01 wt. % to about 3 wt. % natural antimicrobial agent.

21. An antimicrobial skin treatment composition according to claim 20, wherein the natural antimicrobial agent comprises squalane and the composition comprises about 0.1 wt. % to about 1 wt. % squalane.

22. An antimicrobial skin treatment composition according to claim 14, wherein the protease enzyme is coated with a polymer.

23. An antimicrobial skin treatment composition according to claim 14, wherein the composition further comprises about 0.01 wt. % to about 15 wt. % skin healing agent, wherein the skin healing agent comprises at least one of D-alpha tocopheryl acetate, DL-alpha tocopherol acetate, mixed tocopheryls; alantoin; thiocitic acid; or apricot kernal.

24. An antimicrobial skin treatment composition according to claim 14, wherein the composition comprises about 20 wt. % to about 65 wt. % of lower alcohol having 5 carbon atoms or less.

25. A method of producing an antimicrobial composition for application to skin comprising:
heating and mixing a mixture comprising a thickening agent and water to form an emulsion, wherein the thickening agent comprises fatty alcohol having greater than 11 carbon atoms, and
adding to the mixture an antimicrobially effective amount of quaternary ammonium antimicrobial agent.

26. The method of claim 25, wherein the quaternary ammonium antimicrobial agent is mixed with water prior to adding to the mixture.

27. The method of claim 25 wherein the quaternary ammonium component comprises benzethonium chloride.

28. The method of claim 25 wherein the thickening agent and water are mixed at a rate of about 900 rpm or less.

29. The method of claim 25 wherein the mixture is heated and mixed until the emulsion includes micelles having a relatively uniform size.

30. The method of claim 25 wherein the mixture is heated to a temperature between 158 degrees and 210 degrees Fahrenheit at atmospheric pressure.

31. The method of claim 25 wherein an amount of thickening agent is added to the mixture to provide a composition having a viscosity of about 9,000 cps to about 14,000 cps.

32. The method of claim 25 wherein an amount of thickening agent is added to the mixture to provide a composition having a viscosity of about 50 cps to about 500 cps.

33. The method of claim 25 further comprising cooling the emulsion to below the activation temperature of a protease enzyme, and adding the protease enzyme to the emulsion.

34. The method of claim 33 wherein the protease enzyme is coated with a polymer.

35. The method of claim 33 wherein the pH of the emulsion is modified prior to the addition of the protease enzyme to prevent degradation of the protease enzyme.

36. The method of claim 25 further comprising adding to the mixture a natural antimicrobial agent comprising at least one of squalane, licorice root extract, or azealic acid.

37. The method of claim 25 further comprising adding to the mixture a skin healing agent comprising at least one of D-alpha tocopheryl acetate, DL-alpha tocopherol acetate, mixed tocopheryls, alantoin, thiocitic acid, or apricot kernal.

38. The method of claim 25 further comprising adding to the mixture an emollient comprising at least one of silicon, glycerin, petroleum, paraffin, beeswax, lanolin, cod liver oil, elastin, olive oil, coconut oil, avocado oil, castor oil, peanut oil, sesame oil, and almond oil, light mineral oil, vegetable oil, cocoa butter, or alantoin.

39. The method of claim 25 further comprising adding to the mixture a humectant comprising at least one of glycol, propylene glycol, glycerin, sorbitol, urea, alpha hydroxy acid, or sugar.

40. A method for inhibiting the transmission of microbial pathogens comprising:
applying an effective amount of a composition to the skin, and
rubbing the composition into the skin until the composition has been thoroughly dispersed over the skin, wherein said composition comprises:
(a) an antimicrobial effective amount of an antimicrobial package comprising:
(i) quaternary ammonium antimicrobial agent comprising at least one of benzethonium chloride, benzalkonium chloride, methyl benzethonium chloride, or benzoxonium chloride;
(b) thickening agent comprising a fatty alcohol having at least 11 carbon atoms; and
(c) water, wherein the thickening agent are mixed to provide an emulsion.

41. The method of claim 40 wherein the composition provides a three log reduction of gram positive bacteria for at least about 4 hours after application

42. The method of claim 40 wherein the composition provides a three log reduction of gram positive bacteria for at least about 60 hours after application
